(19) United States
(12) Patent Application Publication
(10) Pub. No.: US 2022/0064678 A1
(43) Pub. Date: Mar. 3, 2022

(54) METHOD FOR PRODUCTION OF VITAMIN K USING BIOFILM REACTORS

(71) Applicants: The Penn State Research Foundation, University Park, PA (US); UNIVERSITY OF WAIKATO, Hillcrest (NZ)

(72) Inventors: Ali DEMIRCI, Port Matilda, PA (US); Ehsan MAHDINIA, Albany, NY (US); Aydin BERENJIAN, Hamilton East (NZ)

(21) Appl. No.: 17/312,673

(22) PCT Filed: Dec. 10, 2019
(86) PCT No.: PCT/US2019/065358

§ 371 Date: Jun. 10, 2021

Related U.S. Application Data
(60) Provisional application No. 62/778,141, filed on Dec. 11, 2018.

(51) Int. Cl. C12P 7/66

(52) U.S. Cl. CPC C12P 7/66; C12R 2001/125

(57) ABSTRACT

Provided are improved methods for Vitamin K, including but not necessarily limited to MK-7 production through bacterial fermentation using biofilm reactors. Fed-batch addition of carbon sources, such as glucose, are used as the base media in biofilm reactors. Fed-batch strategies are shown to be significantly effective in glucose-based medium, increasing the end-product concentrations to more than 2-fold higher than the level produced in suspended-cell bioreactors.

### CROSS-REFERENCE TO RELATED APPLICATIONS

This application claims priority to U.S. provisional patent application No. 62/778,141, filed Dec. 11, 2018, the entire disclosure of which is incorporated herein by reference.

### STATEMENT REGARDING FEDERALLY SPONSORED RESEARCH OR DEVELOPMENT

This invention was made with government support under Hatch Act Project No. PEN04561 awarded by the United States Department of Agriculture/NIFA. The government has certain rights in the invention.

### FIELD OF THE DISCLOSURE

This disclosure generally relates to improved approaches to Vitamin K production.

### BACKGROUND

Not long after the discovery of vitamin K as an essential cofactor for blood clotting by Dr. Henrik Dam (Dam 1935), it was discovered that vitamin K comes in two major forms in nature (Widhalm et al. 2012). The plant form, known as phylloquinone, is found abundant in most leafy green vegetables such as spinach and kale (Booth 2012; Binkley et al. 1939). The animal and microbial forms, known as menaquinones have several subtypes (designated MK-1 to MK-15) and include the predominant forms in microbial metabolisms (Mandinia et al. 2017a). The bacterial flora in human intestines do secrete significant amounts of menaquinones, yet due to the very low bioavailability of these sources, no significant absorption takes place (Davidson et al. 1998; Walther et al. 2017). Therefore, microbial fermentation of MK-7 on an industrial scale and supplementing it via diet supplementary pills is one of the only feasible ways to boost vitamin K levels in human metabolism (Berenjian et al. 2015). Menaquinone-7 (MK-7) is the most potent form of all the Vitamin K subtypes that were studied for this purpose with extraordinary benefits for human health (Schurgers et al. 2007; Howard and Payne 2006; Gast et al. 2009; Geleijnse et al. 2004; Yamaguchi 2006).

The most common bacterial strains that were studied are Bacillus subtilis natto (Berenjian et al. 2011a), Bacillus licheniformis (Goodman et al. 1976) and Bacillus amylolyquifaciens (Wu and Ahn 2011). Consequently, B. subtilis natto has been most common in the studies. Both solid state fermentation (SSF) and liquid state fermentation (LSF) strategies have been investigated for MK-7 production with B. subtilis natto (Singh et al. 2015; Wu and Ahn 2011). However, both SSF and static LSF strategies with no robust agitation and aeration (i.e. agitation and aeration rates that would create high enough mass and heat transfer rates to have homogenous conditions), face serious scale-up, and operational issues (Pandey 2003; Mandinia et al. 2017a). Nevertheless, pelicle and biofilm formations that create these issues are beneficial for the MK-7 biosynthesis in the bacteria (Ikeda and Doi 1990). Thus, there is an opportunity to use biofilm reactors to harness the biofilm formations and keep these benefits and at the same time have robust agitation and aeration.

In biofilm reactors, biofilm formations are created through passive immobilization of planktonic cells onto a suitable surface (Kuchma and O'Toole 2000; Demirci et al. 2007; Cheng et al. 2010; Lin et al. 2016). In the past decades, many value-added productions have been enhanced by the use of biofilm reactors (Ercan and Demirci 2013; Izmirlioglu and Demirci 2016; Ho et al. 1997; Khiyami et al. 2006). Using the most potent combination of strain and PCS for MK-7 production (Mandinia et al. 2017b), biofilm reactors have been constructed and utilized to enhance MK-7 production in B. subtilis for batch fermentations for two different media; glycerol and glucose-based media. However, carbon source depletion occurs quite before MK-7 concentrations cease.

In view of the foregoing, there is an ongoing need for modulating carbon source depletion and other parameters that are used for Vitamin K production in a variety of bioreactors. The present disclosure is pertinent to this need.

### SUMMARY

The present disclosure provides a method and system for producing Vitamin K. In embodiments, a method of the disclosure comprises introducing into a biofilm reactor a first glucose or glycerol containing bacteria culture media (e.g., culture media comprising glucose or glycerol). A biofilm comprising bacteria that are capable of producing the Vitamin K forms on a surface in the biofilm reactor. The first glucose or glycerol containing bacteria culture media may be replaced one or more times during formation of the biofilm, such as 1, 2, 3, 4, or more times. Subsequently the method comprises providing introducing a second glycerol or glucose containing bacteria culture media into the biofilm reactor and agitating the bacteria culture medium. Subsequently, the Vitamin K may be separated from the biofilm reactor, and purified to any desired degree of purity.

In embodiments, the first glucose containing bacteria culture media is initially introduced into the biofilm reactor. In embodiments, the first glucose containing bacteria culture media comprises about 150 g/L of the glucose. “About” as used herein with respect to glucose or glycerol containing media means ±2g/L.

In embodiment, the method further comprises introducing a second glycerol or glucose containing bacteria culture media into the biofilm reactor. The second glycerol or glucose containing bacteria culture media maybe introduced at about 72 hours after the introduction of the first glucose containing bacteria culture media. “About” with respect to hourly time periods as used herein means±one hour. In an embodiment, the second glycerol containing bacteria culture media is introduced into the biofilm reactor. In an embodiment, the second glycerol containing bacteria culture media comprises about 45 g/L of the glycerol.

In an embodiment, a method of this disclosure further comprises introducing a third glycerol containing bacteria culture media into the biofilm reactor. The third glycerol containing bacteria culture media may be introduced at about 144 hours after the introduction of the first glycerol containing bacteria culture media. In an embodiment, the third glycerol containing bacteria culture media comprises about 45 g/L of the glycerol.

In another embodiment, instead of glycerol containing media, the method comprises introducing a second glucose containing bacteria culture media into the biofilm reactor. In an embodiment, the second glucose containing bacteria culture media comprises about 150 g/L of the glucose. In an embodiment, a method of the disclosure further comprises introducing a third glucose containing bacteria culture media into the biofilm reactor. The third the third glucose containing media may be introduced at about 144 hours after the introduction of the first glucose containing bacteria culture media.

In embodiments, at least 24 mg/L of the Vitamin K is produced. In embodiments, at least 24 mg/L of the Vitamin K is produced over a time period of not more than about 288 hours from introducing the first glucose or glycerol containing bacteria culture media into the biofilm reactor. In embodiments, from 24-30 mg/L, inclusive, and including all ranges of integers there between of the Vitamin K is produced. In embodiments, from 28-29 mg/L of the Vitamin K is produced. In embodiments, at least 24 mg/L of the MK-7 is produced over a time period of not more than about 288 hours from introducing the first glucose or glycerol containing bacteria culture media into the biofilm reactor.

In embodiments, the agitating is performed continuously over some or the entire period of Vitamin K production. In one embodiment, the agitating is performed for a period of about 288 hours.

In embodiments, Vitamin K produced by a method of the disclosure comprises or consists of Menaquinone-7 (MK-7).

In embodiments, the Vitamin K is produced in the biofilm reactor by bacteria that are Bacillus subtilis. The bacteria may be Bacillus subtilis natto.

In embodiments, the biofilm reactor used in a system or method of the disclosure comprises one or a plurality of plastic composite supports (PCS) that increase surface area on which the biofilm is formed, and may also serve to attract planktonic bacteria cells to migrate to the PCS. The PCS may be coated or impregnated with bacteria nutrients.

In an embodiment, the disclosure provides preparation of purified Vitamin K produced according to a method of the disclosure. The Vitamin K may comprise or essentially consist of or consist of MK-7.

In another aspect, the disclosure provides a system comprising a biofilm reactor and a plurality of PCS, the plurality of PCS supports comprising a biofilm comprising bacteria that produce Vitamin K. The biofilm reactor may further comprise a bacterial culture media comprising glucose, glycerin, or a combination thereof. The system is configured such that bacterial culture media comprises at least 24-30 mg/L of the Vitamin K. The bacteria in the system may comprise Bacillus subtilis, which may be Bacillus subtilis natto.

### BRIEF DESCRIPTION OF FIGURES

FIG. 1: MK-7 biosynthesis in glucose and glycerol-based media with different concentrations of glucose or glycerol as fed-batch implementations through 144 h of fermentation.

FIG. 2: Maximum MK-7 profile in glucose-based medium obtained with 150 g/L glucose solution fed-batch added at 72 h of fermentation.

FIG. 3: Maximum MK-7 profile in glycerol-based medium obtained with 30 g/L glycerol solution fed-batch added at 72 h of fermentation.

FIG. 4: Maximum MK-7 concentrations after 288 hours of fermentation in glucose (starting with 150 g/L) and glycerol-based (starting with 45 g/L) media with different fed-batch strategies implemented.

FIG. 5: Highest MK-7 concentration profiles observed in the glucose-based medium with the optimum starting composition of 152.6 g/L glucose at 30° C., pH 6.48 and 234 rpm agitation.

FIG. 6: Highest MK-7 concentration profile observed in the glycerol-based medium 45 g/L glycerol at 35° C., pH 6.60 and 200 rpm agitation.

FIG. 7: Morphology change in B. subtilis cells going from 24-h (A) to 144-h (B) and 288-h (C) in the glucose-based medium and 24-h (D) to 144-h (E) and finally 288-h (F) in the glycerol-based medium.

FIG. 8: SEM images of the interior and the exterior of the PCS where (A) shows the interior of the control PCS at 60× magnification and (A′) is the 10,000× magnification of the square region in (A). Similarly, (B) and (B′) show the exterior of the control; (C) and (C′) show the interior and (D) and (D′) show the exterior of the PCS in glucose-based medium, (E) and (E′) show the interior and (F), (F′) (5,000×) and (F″) (10,000×) show the exterior of the PCS in glycerol-based medium. (F″′) is a close-up view of a single B. subtilis cell attached to the exterior surface of the PCS via the γ-polyglutamate (γ-PGA) extracellular matrix at 80,000× magnification.

### DETAILED DESCRIPTION OF THE DISCLOSURE

Unless defined otherwise herein, all technical and scientific terms used in this disclosure have the same meaning as commonly understood by one of ordinary skill in the art to which this disclosure pertains.

Every numerical range given throughout this specification includes its upper and lower values, as well as every narrower numerical range that falls within it, as if such narrower numerical ranges were all expressly written herein.

All steps, and all combinations of steps described herein are used, and can be used consecutively, or the order of the steps can be varied. Any particular step, or combination of steps, can be excluded from the claimed process.

Any value or measurement described herein can be compared to any other value, such as a reference value, a control, or any other measurement, number, quality or characteristic. In certain embodiments, an amount, purity, enrichment, etc., or pace of production of a product, such as Vitamin K, including but not necessarily limited to Menaquinone-7 (MK-7), produced by a process described herein, is increased relative to a reference value. In embodiments, the reference value is a MK-7 amount produced using a variation in at least one parameter that is a component of a process described herein. Such parameters include but are not necessarily limited to the type/amount of bacteria used in a Vitamin K fermentation production process, periods of time, such as any period of time that pertains to bacterial growth and/or function, such as during fermentation, including but not limited to stationary and lag phase fermentation, temperature, heat transfer, biofilm formation and biofilm characteristics, growth and/or morphological characteristics of bacteria in a fermentation vessel and/or a biofilm, such as the amount of bacteria within a biofilm and in a planktonic form, biofilm density, location of the bacteria in the fermentation vessel, volume and/or type of bacteria growth medium, changes in spores and/or sporulation, use and/or order of reagent addition and/or utilization, including but not limited to nitrogen sources, carbon sources, such as glucose and glycerol, degree and rate of consumption of nitrogen and carbon sources, amounts and rates of Vitamin K secretion, agitation, shear forces, and other parameters that will be apparent to those skilled in the art, given the benefit of this disclosure. In an embodiment, production of Vitamin K is increased using a biofilm reactor, relative to a production in a suspended-cell bioreactor. In embodiments, Vitamin K production is increased when glucose is used as a carbon source, and/or a first carbon source. In a non-limiting embodiment, Vitamin K is increased relative to using glycerol as a carbon source during Vitamin K production. In embodiments, Vitamin K is produced in an essentially glycerol free medium. In embodiments, glucose is the only carbon source added to a biofilm bioreactor. In embodiments, the glucose is batch fed. In embodiments, only two, or at least two, additions of a carbon source such as glucose are used during the production of Vitamin K.

The disclosure includes the vessels described herein, including the vessels in all stages of operation. In embodiments, the vessels are biofilm bioreactors. In embodiments, the bioreactors have a volume of a least 2 liters. In embodiments, a bioreactor used in a process of this disclosure has a volume that is from 2 to up to 3 million liters, inclusive, and include all numbers and ranges of numbers there between.

In embodiments, bacteria used in methods of this disclosure are any bacteria capable of producing Vitamin K, and in particular are capable of producing MK-7. In embodiments, the bacteria are any of the Bacillus bacteria. In embodiments, the bacteria are Bacillus subtilis. In embodiments, the bacteria are Bacillus subtilis natto, Bacillus licheniformis or Bacillus amylolyquifaciens.

Non-limiting examples of the disclosure are described below. The examples were performed in 2-liter bench-top bioreactors to demonstrate feasibility in larger fermenters (˜100 L) on industrial scales. Therefore, in embodiments, the disclosure includes starting a Vitamin K production process with glucose to facilitate metabolism, and subsequently switching to glycerol fed-batch additions, which are less expensive and more sustainable than glucose.

The following examples are intended to illustrate various embodiments but not limit the disclosure.

### EXAMPLE 1

The following materials and methods were used to produce the results described in the Examples.

### Materials and Methods

### Microorganisms and Media

Bacillus subtilis natto (NF1) was isolated from commercial natto (Mizkan Co., Ltd., Handa, Japan), as previously described (Mandinia et al. 2017b, from which the description is incorporated herein by reference). For biofilm formation on the Plastic Composite Support (PCS), Tryptic Soy Broth (TSB) medium fortified with 10% (w/v) glucose (Tate & Lyle, Decatur, Ill.) and 0.8% yeast extract (Biospringer, Milwaukee, Wis.) was used. Glycerol-based medium consisted of 10 g of soytone (Difco, Detroit, Mich.)), 5 g of yeast extract (Difco), 45 g of glycerol (EMD Chemicals, Gibbstown, N.J.) and 0.6 g of K₂HPO₄ (VWR, West Chester, Pa.) and the glucose-based medium consisted of TSB 0.8% yeast extract (Biospringer) and 150 g/L glucose (Tate & Lyle) per liter of deionized water.

### Biofilm Reactors

Sartorius Biostat B Plus twin system bioreactors (Allentown, Pa.) equipped with 2-L vessels (1.5-L working volume) were utilized. Sterile 4N sulfuric acid (EMD) and 4N sodium hydroxide (Amresco, Solon, Ohio) along with antifoam B emulsion (Sigma-Aldrich, Atlanta, Ga.) were added automatically to maintain pH and suppress foaming as needed. Plastic Composite Support (PCS) tubes type SFYB (50% Polypropylene, 35% soybean hulls, 5% soybean flour, 5% yeast extract, 5% bovine albumin and salts) were manufactured and implemented (65 mm×10.5 mm tubes) and biofilm reactors for glycerol and glucose-based media were operated at optimum aeration (1 vvm), agitation (200 rpm for glycerol and 234 rpm for glucose), pH (6.48 for glucose and 6.6 for glycerol) and temperatures (30° C. for glucose and 35° C. for glycerol) as described in previous studies (Ho et al. 1997;

### Biofilm Formation

For biofilm formations to form on the PCS grids, bioreactors were set up with grid-like fashion PCS formations. Then, sterile medium was added to the bioreactors and replenished for four repeated fermentation cycles. At the end of the four fermentation cycles, the fermentation broth was sampled and Gram-stained to verify suspended-cell culture purity.

### Experimental Design

After the biofilm reactors were in operation, fed-batch fermentation runs were started with main fermentation media. Then, sterile glycerol solutions for 15, 30 or 45 g/L additions were prepared in 150 mL of total volume feeding and glucose for 50, 100 or 150 g/L additions solutions were prepared in 400 mL of total volume feeding at 72 and 144 h of fermentation. Glucose and glycerol additions were implemented in glucose or glycerol-based media to investigate cross-effects. Samples were obtained every 12 hours until 288 hours for MK-7 and substrate analysis.

### Analysis

(i) MK-7 Analysis

Three mL of fermentation broth was mixed with 2:1, (v/v) n-hexane:2-propanol mixture to extract the MK-7 content (Berenjian et al. 2011b). N-hexane:2-propanol (2:1, v/v) with 1:4 (liquid:organic, v/v) was used. The mixture was vigorously shaken using a vortex mixer for 3 min and then the organic phase was separated and evaporated under forced air flow at ambient temperature. Then, dried pellets containing the MK-7 were dissolved in methanol in a Biosonic ultra-sonication water bath (Cuyahoga Falls, Ohio) for 15 min at ambient temperature. After the pellets were completely suspended in methanol, the mixtures were filtered through 0.2 μm PTFE filters (PALL Life Sciences, Port Washington, N.Y.). MK-7 concentrations in the samples were then analyzed by High Performance Liquid Chromatography (HPLC) using UV-Vis light (248 nm).

(ii) Substrate Analysis

Samples of the fermentation broth was centrifuged at 9000×g for 5 min (Microfuge 20 Series, Beckman Coulter Inc., Brea, Calif.) and then filtered through 0.2 μm cellulosic filters (PALL). Then, with no dilution, the cell-free broth was analyzed by HPLC using a refractive index (RI) detector for glucose and/or glycerol concentrations as described in (Mandinia et al. 2018a; 2018b; 2019a, the descriptions of each of which are incorporated herein by reference).

(iii) Statistical Analysis

All observations were repeated and the average values were obtained and demonstrated with standard errors of the repetitions as error bars. Using Minitab 17.0 ANOVA (Minitab Inc., State College, Pa.), any difference with p<0.05 was considered significant.

### Light Microscopy

After Gram staining, B. subtilis cells from glucose and glycerol-based medium fermentations in biofilm reactors at ages of 28 h, 144 h and 288 h were observed using a ZEISS Axio Scope Imager Alm light microscope equipped with an AxioCam MRm camera (ZEISS, Ontario, Calif.).

### Scanning Electron Microscopy

Scanning Electron Microscopy (SEM) was utilized to observe and evaluate biofilm formation on the PCS tubes in comparison with the control before cell growth. The biofilm cells on the exterior and interior surfaces of the PCS tubes were maintained by chemical fixation of the cells. PCS tubes were soaked in 2.5% gluteraldehyde in 0.1M phosphate buffer (pH 7.2) with 0.02% Triton X-100. Then the fixative solution was decanted and samples were washed 3-5 times with the phosphate buffer and then were serially dehydrated with 25, 50, 70, 85, 95, and 100% (×3) ethanol for 5 min. Finally, the remaining moisture was eliminated using critical point drying for 3 hours. Zeiss Sigma Variable Pressured Field Emission Electron Scanning Microscope (VP-FESEM, ZEISS, Ontario, Calif.) was used to observe the processed surfaces (Pashazanusi et al. 2017; Izmirlioglu and Demirci 2017).

### EXAMPLE 2

For fed-batch fermentation in biofilm reactors, the target substrate concentrations are included in amounts sufficient to maintain the stationary phase as long as desired and at the same time the concentrations do not have inhibitory and negative effects (Mandinia et al., 2019b). For these purposes, several different combinations of fed-batch additions were implemented, as follows.

### Effects of Carbon Source on Fed-Batch Fermentations

(i) Carbon Source Concentration

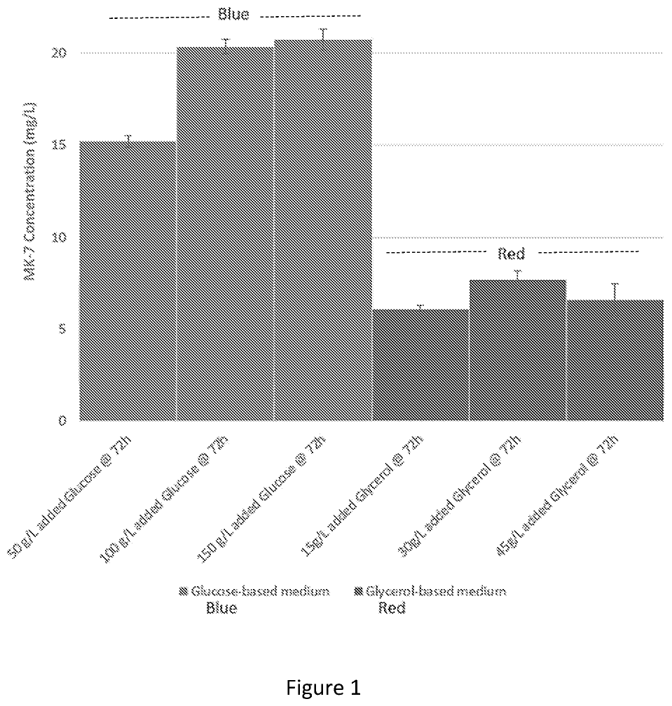

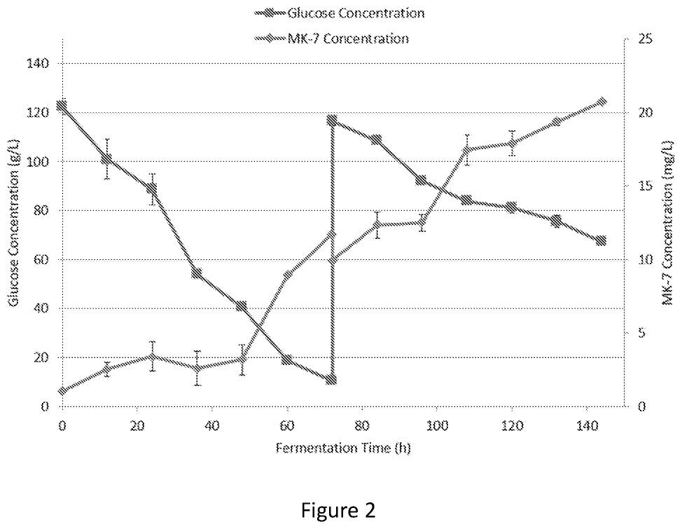

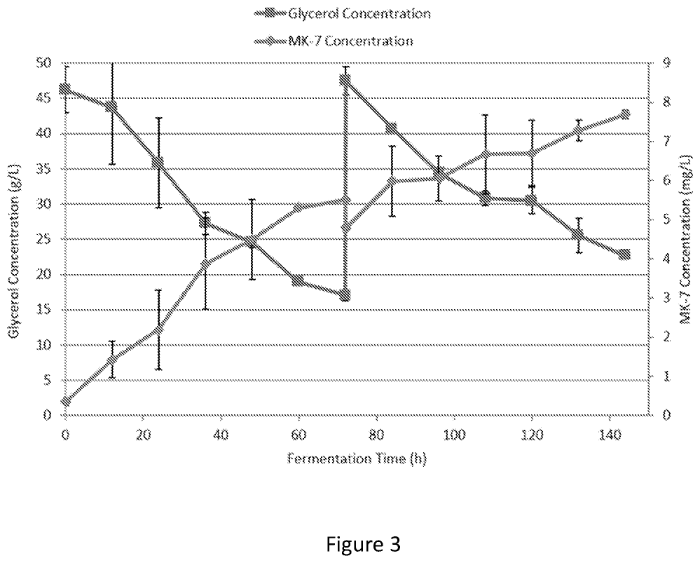

The optimum glucose-based medium starts the fermentation with an initial 150 g/L of glucose and the optimum glycerol-based medium starts with 45 g/L glycerol. Higher concentrations for glucose and glycerol may result in severe inhibition of MK-7 secretion (Mandinia et al. 2018c; 2018d; 2019c, the descriptions of each of which are incorporate herein by reference); the fed-batch concentrations that were designed in this disclosure targeted equal or less amounts of the starting concentrations. In other words, 50, 100 and 150 g/L glucose and 15, 30 and 45 g/L glycerol were the compositions that were implemented to evaluate the most efficient strategy (FIG. 1). Carbon source depletion typically occurs on or around 72 h of fermentation in both media (FIGS. 2 and 3). Glucose consumption in biofilm reactors happened more quickly at around 95% of the initial glucose is consumed within the first 72 h (FIG. 2); whereas for glycerol, at 72 h still over 35% of the initial glycerol still exists in the broth (FIG. 3).

### EXAMPLE 3

(ii) Glucose-Based Medium

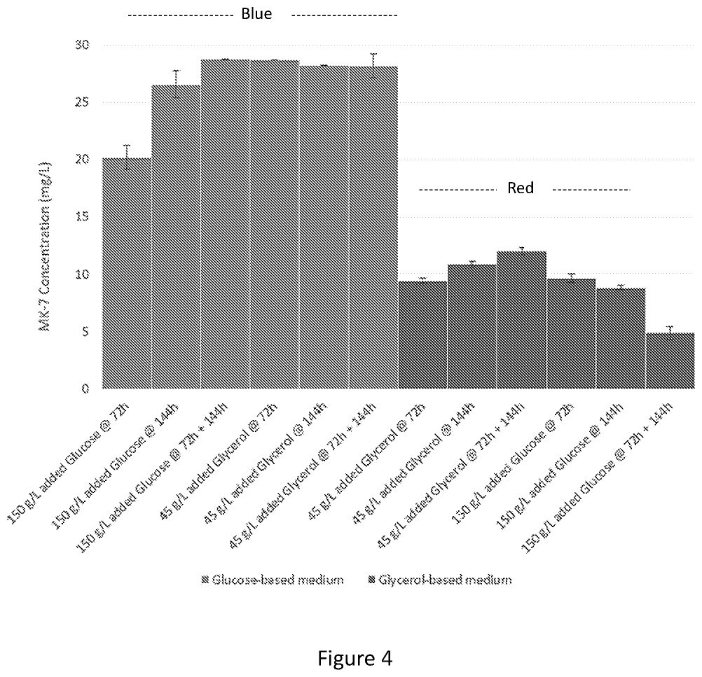

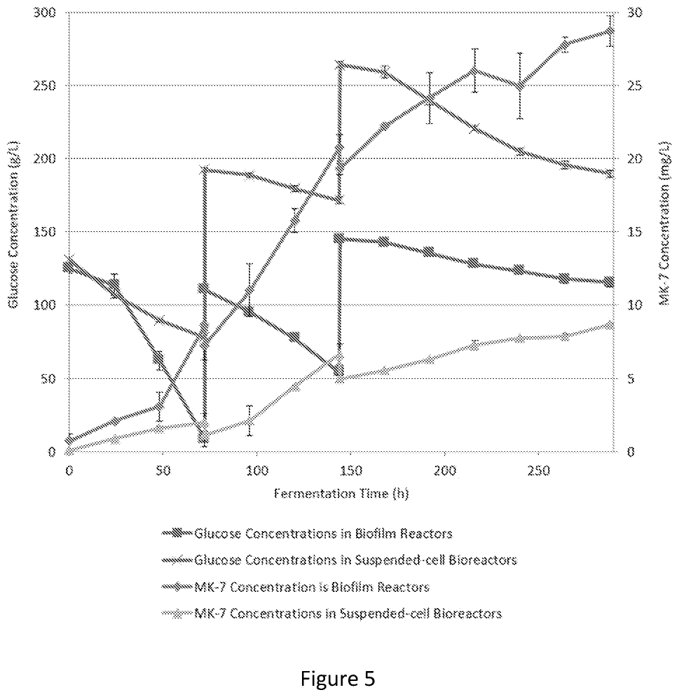

Using the 150 g/L glucose injection at 72 h into the glucose-based medium with only this injection and continuing the fermentation until 288 h, the added glucose was not adequate to maintain the fermentation in suitable conditions of stationary phase for sufficient time, and therefore the MK-7 profile plateaued and did not exceed 21 mg/L boundary (FIG. 4). Conversely, feeding at 144 h yielded improved results (26.5±1.8 mg/L) despite the glucose depletion that occurred between 72 h and 144 h and that glucose was depleted by 288 h (data not shown). Improved results were obtained by feeding at 72 and 144 h, which led to 28.7±0.3 mg/L MK-7 concentration (FIG. 5). This was the highest amount observed in bioreactors, and thus the production in biofilm reactors is comparable to the maximum concentrations in static fermentations (32.5±0.4 mg/L) (Mandinia et al. 2018d, 2019d, 2019e). Also, these concentrations in biofilm reactors were 230% higher than the concentrations achieved in suspended-cell reactors with the same conditions (8.7±0.2 mg/L). Thus, the disclosure demonstrates significant improvements achievable by biofilm reactors for MK-7 production (FIG. 5).

### EXAMPLE 4

(iii) Glycerol-Based Medium

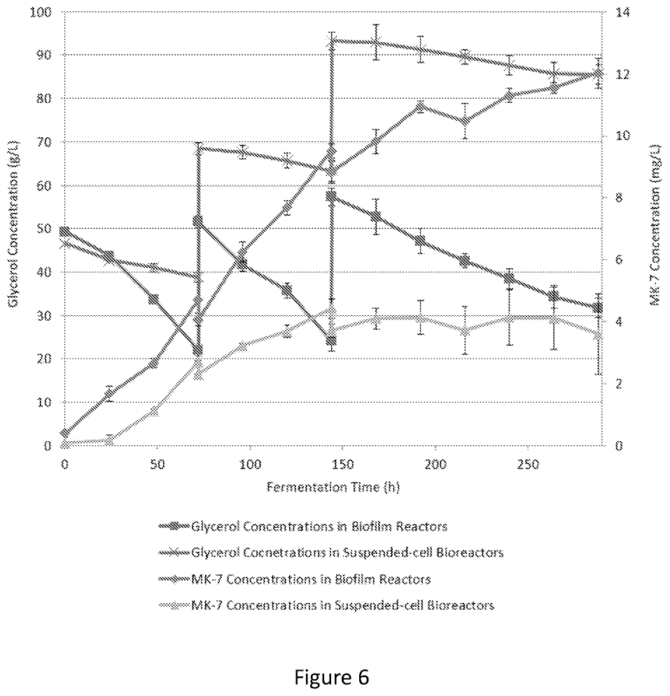

When fed-batch strategies were used for glycerol-based medium, inhibitory effects were readily observable throughout all the experiments. As can be seen in FIG. 4, similar to the glucose-based medium, the double feeding approach (72 and 144 h) was better than single feeding at 72 h. Without intending to be bound by any particular theory, it is considered this may be due to inhibitory effects being overcome by the positive effect of starvation inhibition. On the other hand, glucose feeding into the glycerol-based medium followed a reverse pattern. As shown in FIG. 4, the double glucose feeding produced the lowest MK-7 concentrations. Therefore, not only did the high glucose levels not appear to induce MK-7 secretion and redeem the inhibitory effects, it appears they amplify them. Furthermore, the highest concentrations achieved with these various fed-batch regimes are 12.0±0.5 mg/L, which is again lower than the 14.7±1.4 mg/L achieved in optimized batch biofilm reactor with glycerol-based medium (Mandinia et al. 2018d, the description of which is incorporated herein by reference). Thus, unlike the glucose-based medium, fed-batch strategies do not appear to be beneficial for MK-7 production in the glycerol-based medium in biofilm reactors; despite the more robust metabolism observed in fed-batch biofilm reactors compared to suspended-cell reactors (FIG. 6). Production in suspended-cell reactors were also inhibited by glycerol presence; however, the gap between the concentrations in suspended-cell and biofilm reactors were still significant as the plateau in the profile in suspended-cell reactors suggest inhibition from a very early stage (FIG. 6).

### EXAMPLE 5

### Morphology Studies

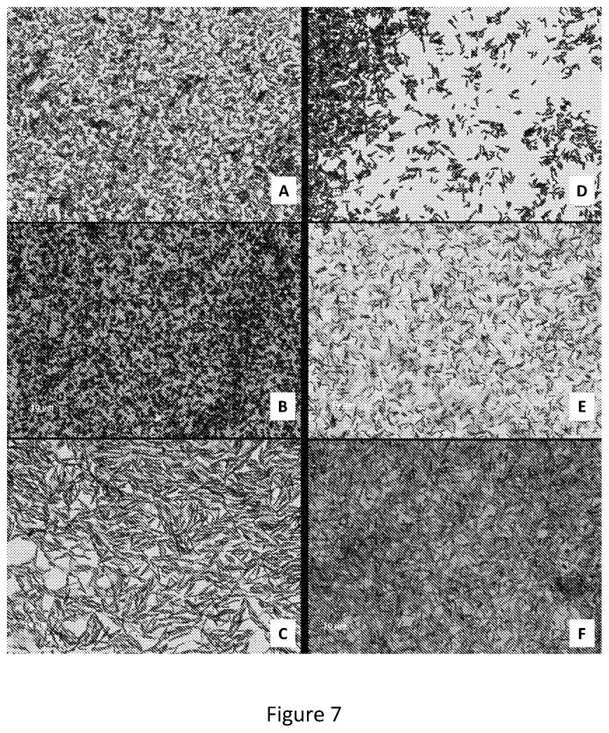

B. subtilis cells morphed and changed during the long 12 days of fermentation. FIG. 7 clearly indicates that as fermentation process in both media, young short cells (typically 2 μm long) that are observed at 24-h morph into long aged Bacilli cells (that are sometimes about 10 μm long).

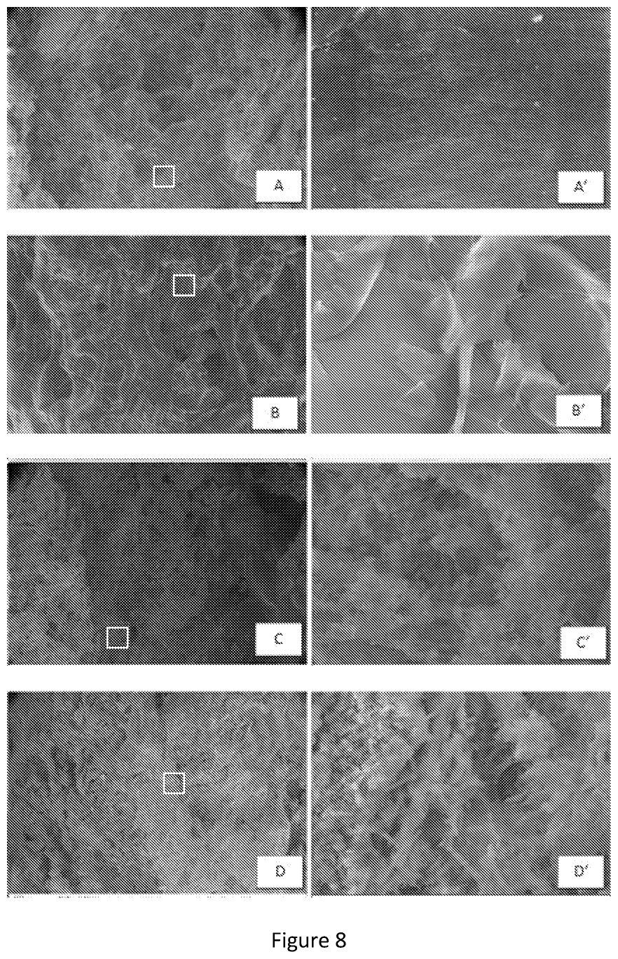

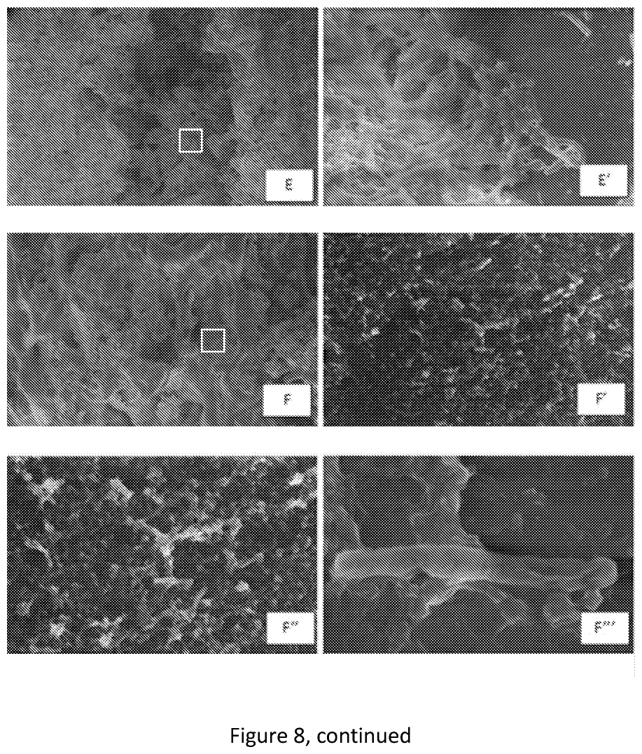

In order to confirm the formation of biofilms on the PCS and to investigate the morphology on the PCS with the extracellular matrices, SEM was used. FIG. 8 shows the low magnification captures of the PCS surfaces (60× magnifications) and the higher magnifications to observe the cells in the biofilm forms (10000× magnifications). These clearly showed that immobilized cells in biofilm formations existed on the surface of the PCS tubes in both media (FIG. 8 panel D′ and panel F″). Even some robust biofilm population was found inside the tube center hole when the tube was cut (FIG. 8C′ and E′). In the glucose-based media, the biofilm density on the surface was significantly more than the center of the tube of course. However, this was not the case for the PCS in the other medium, where the biofilm observed in the tube center was comparable and in some cases even more robust than the ones on the surface.

In view of the foregoing examples, the following will be recognized.

Since glucose is a preferred and more readily metabolized carbon source compared to glycerol by B. subtilis strains, the consumption rate for glucose is markedly higher (Stulke and Hillen 1999). Accordingly, as evident in FIGS. 2 and 3, in both cases carbon source consumption is efficiently continued after the injections; yet in the case of glucose, consumption continues with a steeper slope (max 2.83 g/L/h) compared to glycerol (max 0.67 g/L/h). As FIG. 1 indicates, for glucose, the MK-7 profile ascends with glucose concentration, and the final MK-7 concentration of 20.7±1.2 mg/L achieved with 150 g/L glucose supplementation is consistent with the 20.5±0.5 mg/L maximized concentrations in batch biofilm reactors (Mandinia et al. 2018c). Thus, and without intending to be constrained by any particular theory, glucose does not appear to exert inhibitory effects on MK-7 profile and therefore, the higher concentration of 150 g/L is favorable. On the other hand, when glycerol was applied in the glycerol-based medium, a different result was obtained. In particular, as seen by FIG. 1, the middle concentration of 30 g/L is distinct compared to 15 and 45 g/L, which suggests an inhibitory effect. Furthermore, the highest concentration achieved in this case was 7.7±1.1 mg/L, which is significantly lower than the concentrations achieved in batch biofilm reactors (14.7±1.4 mg/L) (Mandinia et al. 2018d). This observation also suggests a glycerol inhibitory effect in the glycerol-based medium. Glycerol is believed to have beneficial effects on MK-7 secretion, and fed-batch glycerol addition in shake-flasks has been more successful where fed-batch addition of glycerol at 48 h increased the final MK-7 concentrations by about 40% (86.5±0.5 mg/L after extraction) (Berenjian et al. 2012; Berenjian et al. 2011a). However, in biofilm reactors with fed-batch fermentation, it appears that the inhibitory effects persist in the same manner as in batch biofilm reactors (Mandinia et al. 2018d). As a result, the middle concentration of 45 g/L in consistence of the initial concentration was elected.

Single and double injections of glycerol solutions into the glucose-based medium led to similar results (28.6±0.1, 28.2±0.1 and 28.1±1.2 mg/L). Without intending to be bound by any particular theory, it is considered that the reason for this may be that MK-7 is a mixed metabolite; it begins in the exponential phase and continues as long as severe starvation does not occur and fermentation is maintained in the stationary phase. While the glycerol-based medium cannot support a metabolism as robust as the glucose-based version, glycerol fed-batch additions are adequate to preserve the fermentation in stationary phase long enough to reach these high concentrations. It is considered that some of the benefits of glycerol presence might have arisen without inhibiting the secretion. As FIG. 5 shows, the second feeding is slowly consumed with around 190 g/L glucose remaining at the end of the 12-day fermentation period, while the first feeding is consumed more rapidly, and the initial concentrations are consumed even faster.

It is known that B. subtilis species are potent spore former strains and sporulation is triggered by N-source starvation (Fisher 1999). It is also known that sporulation and morphological and consequent gene expression changes are closely connected (Stragier et al. 1988). Since the only carbon and nitrogen sources used in this disclosure were supplied only in the beginning of the fermentation, nitrogen starvation may be possible, which along with passive immobilization and biofilm formation can lead to such adaptations in morphology. Another explanation could be that biofilm reactors are based on passively immobilized cells on the PCS that initiate the planktonic population in each batch. Also, biofilm reactors in this case are highly agitated and the shear stress on the PCS is considerable. Thus, it is possible that the short tough cells are arising from the PCS-based biofilm, which are adapted to endure the stress, and as these planktonic cells reproduce away from that stress in the following generations, the need to be short and tough goes away and the long relaxed cells are replaced.

The substantial biofilm population in the center of the PCS tubes in the glycerol-based medium was surprising since B. subtilis is highly aerobic and tends to stay on the surface where dissolved oxygen is more available, unlike anaerobic microorganisms which may prefer to seep inside for more anaerobic conditions (Izmirlioglu and Demirci 2017). However, one explanation could be that the less nourishing glycerol medium could not enable the cells to strive on the surface and handle the physical stress as well as the glucose-based medium. It is hard to miss how much denser the biofilm formations are in glucose-based medium (FIG. 8D′) compared to the ones formed on the surface of the PCS in the glycerol-based medium (FIG. 8F″). Also, the γ-polyglutamate extracellular matrices depositions are also clearly visible and also distinct in the two media and of course in comparison with the surface of the control (FIGS. 8A′ and B′). Finally, FIG. 8F′ (5,000× magnification) shows how the matrices look like containing the less populated cells and FIG. 8F″′ shows a close-up morphology of a singular B. subtilis cell attached onto the matrix.

As carbon source depletion occurs in both glycerol and glucose-based media in B. subtilis fermentation, the present disclosure includes increasing final MK-7 concentrations by applying fed-batch carbon source additions. Results of this disclosure show that in glucose-based medium, double glucose feeding yields MK-7 production that is the highest concentration reported in bioreactors and was significantly higher than the concentrations in suspended-cell bioreactors under the same conditions. This is a significant step towards the introduction of biofilm reactors as a replacement for current fermentation strategies, including static fermentation strategies, which are difficult to scale up and are associated with mass and heat transfer challenges, and suspended-cell bioreactors which, as demonstrated herein, are not as efficient in MK-7 production as biofilm reactors.

Although the present disclosure has been described with respect to one or more particular embodiments and/or examples, it will be understood that other embodiments and/or examples of the present disclosure may be made without departing from the scope of the present disclosure.

References—This listing of references is not an indication that any of the references are material to patentability.
Berenjian A, Mahanama R, Kavanagh J, Dehghani F (2015) Vitamin K series: current status and future prospects. Crit Rev Biotechnol 35:199-208. doi: 10.3109/07388551.2013.832142
Berenjian A, Mahanama R, Talbot A, Biffin R, Regtop H, Kavanagh J, Dehghani F (2011a) The Effect of Amino-acids and Glycerol Addition on MK-7 Production. Proc World Congr Eng Comput Sci; II.
Berenjian A, Mahanama R, Talbot A, Biffin R, Regtop H, Valtchev P, Kavanagh J, Dehghani F (2011b) Efficient Media for High Menaquinone-7 Production: Response Surface Methodology Approach. N Biotechnol 28:665-672. doi: 10.1016/j.nbt.2011.07.007
Berenjian A, Mahanama R, Talbot A, Regtop H, Kavanagh J, Dehghani F (2012) Advances in Menaquinone-7 Production by Bacillus subtilis natto: Fed-batch Glycerol Addition. Am J Biochem Biotechnol 8:105-110. doi: 10.3844/ajbbsp.2012.105.110
Binkley S B, Maccorquodale D W, Thayer A, Doisy E A (1939) The Isolation of Vitamin K1. J Biol Chem 130:219-234.
Booth S L (2012) Vitamin K: food composition and dietary intakes. Food Nutr Res 56(1):5505. doi: 10.3402/fnr.v56i0.5505
Cheng K C, Demirci A, Catchmark J M (2010). Advances in biofilm reactors for production of value-added products. Applied microbiology and biotechnology 87(2):445-56. doi: 10.1007/s00253-010-2622-3
Dam H (1935) The Antihaemorrhagic Vitamin of the Chick: Occurrence and Chemical Nature. Nature 135:652-653.
Davidson R T, Foley A L, Engelke J A, Suttie J W (1998) Conversion of Dietary Phylloquinone to Tissue Menaquinone-4 in Rats is not Dependent on Gut Bacteria. J Nutr 128:220-223.
Demirci A, Pongtharangkul T, Pometto III A L (2007) Applications of Biofilm Reactors for Production of Value-added Products by Microbial Fermentation. Blackwell Publishing and The Institude of Food Technologists, Iowa, USA.
Ercan D, Demirci A (2013) Production of human lysozyme in biofilm reactor and optimization of growth parameters of Kluyveromyces lactis K7. Appl Microbiol Biotechnol 97:6211-6221. doi: 10.1007/s00253-013-4944-4
Fisher S H (1999) MicroReview Regulation of nitrogen metabolism in Bacillus subtilis: Mol Microbiol 32:223-232. doi: 10.1046/j.1365-2958.1999.01333.x
Gast G C M, de Roos N M, Sluijs I, Bots M L, Beulens J W J, Geleijnse J M, Witteman C J, Grobbee D E, Peeters P H M and van der Schouw Y T (2009). A High Menaquinone Intake Reduces the Incidence of Coronary Heart Disease. Nutr Metab Cardiovasc Dis, 19(7), 504-510. http://doi.org/10.1016/j.numecd.2008.10.004
Geleijnse J M, Vermeer C, Grobbee D E, Schurgers L J, Knapen M H J, van der Meer I M, Hofman A and Witteman J C M (2004). Dietary Intake of Menaquinone is Associated with a Reduced Risk of Coronary Heart Disease: The Rotterdam Study. J Nutr, 134(11), 3100-3105.
Goodman S R, Marrs B L, Narconis R J, Olson R E (1976) Isolation and description of a menaquinone mutant from Bacillus licheniformis. J Bacteriol 125:282-289.
Ho K L, Pometto A L, Hinz P N, Dickson J S, Demirci A (1997) Ingredient Selection for Plastic Composite Supports for L-(1)-Lactic Acid Biofilm Fermentation by Lactobacillus casei subsp. rhamnosus. Appl Environ Microbiol 63:2516-2523.
Howard L M, Payne A G (2006) Health Benefits of Vitamin K2: A Revolutionary Natural Treatment for Heart Disease And Bone Loss, 1st edn. Basic Health Publications, Inc., Laguna Beach.
Ikeda H, Doi Y (1990) A vitamin-K2-binding factor secreted from Bacillus subtilis. Eur J Biochem 192:219-224. doi: 10.1111/j.1432-1033.1990.tb19218.x
Izmirlioglu G, Demirci A (2016) Ethanol production in biofilm reactors from potato waste hydrolysate and optimization of growth parameters for Saccharomyces cerevisiae. Fuel 181:643-651. doi: 10.1016/j.fuel.2016.05.047
Izmirlioglu G, Demirci A (2017) Simultaneous saccharification and fermentation of ethanol from potato waste by co-cultures of Aspergillus niger and Saccharomyces cerevisiae in biofilm reactors. Fuel 202:260-270. doi: 10.1016/j fue1.2017.04.047
Khiyami M A, Pometto A L, Kennedy W J (2006) Ligninolytic Enzyme Production by Phanerochaete chrysosporium in Plastic Composite Support Biofilm Stirred Tank Bioreactors. J Agric Food Chem 54:1693-1698. doi: 10.1021/jf0514241
Kuchma S L, O'Toole G A (2000) Surface-induced and biofilm-induced changes in gene expression. Curr Opin Biotechnol 11:429-33. doi: 10.1016/S0958-1669(00)00123-3
Lin S P, Liu C T, Hsu K D, Hung Y T, Shih T Y, Cheng K C (2016) Production of bacterial cellulose with various additives in a PCS rotating disk bioreactor and its material property analysis. Cellulose 23(1):367-77. doi: 10.1007/s10570-015-0855-0
Mandinia E, Demirci A, Berenjian A (2017a) Production and application of menaquinone-7 (vitamin K2): a new perspective. World J Microbiol Biotechnol 33:2. doi: 10.1007/s11274-016-2169-2
Mandinia E, Demirci A, Berenjian A (2017b) Strain and plastic composite support (PCS) selection for vitamin K (Menaquinone-7) production in biofilm reactors. Bioprocess Biosyst Eng 40:1507-1517. doi: 10.1007/s00449-017-1807-x
Mandinia E, Demirci A, Berenjian A (2018a) Optimization of Bacillus subtilis natto growth parameters in glycerol-based medium for vitamin K (Menaquinone-7) production in biofilm reactors. Bioprocess Biosyst Eng 41:195-204. doi: 10.1007/s00449-017-1857-0
Mandinia E, Demirci A, Berenjian A (2018b) Utilization of glucose-based medium and optimization of Bacillus subtilis natto growth parameters for vitamin K (menaquinone-7) production in bio film reactors. Biocatal Agric Biotechnol., 13:219-224. doi: 10.1016/j.bcab.2017.12.009
Mandinia E, Demirci A, Berenjian A (2018c) Enhanced vitamin K (menaquinone-7) production by Bacillus subtilis natto in biofilm reactors by optimization of glucose-based medium. Curr. Pharm. Biotechno., 19(11): 917-924. doi: 10.2174/1389201020666181126120401
Mandinia E, Demirci A, Berenjian A (2018d) Implementation of fed-batch strategies for vitamin K (menaquinone-7) production by Bacillus subtilis natto in biofilm reactors. Appl. Microbiol. Biot., 102(91): 9147-9157. doi: 10.1007/s00253-018-9340-7
Mandinia E, Demirci A, Berenjian A (2019a) Biofilm reactors as a promising method for vitamin K (menaquinone-7) production. Appl. Microbiol. Biot., 103(14): 5583-5592. doi: 10.1007/s00253-019-09913-w
Mandinia E, Cekmecelioglu D, Demirci A (2019b) Bioreactor scale-up. in: Berenjian, A. (Ed.) Essentials in fermentation technology. Learning materials in biosciences. Springer, Cham, 213-236. doi: 10.1007/s00253-019-09913-w
Mandinia E, Demirci A, Berenjian A (2019c) Effects of medium components in glycerol-based medium on vitamin K (menaquinone-7) production by Bacillus subtilis natto in biofilm reactors. Bioproc. Biosyst. Eng., 42:223-232. doi: 10.1007/s00449-018-2027-8
Mandinia, E., Demirci, A. & Berenjian, A. (2019d): Modeling of vitamin K (menaquinone-7) fermentation by Bacillus subtilis natto in biofilm reactors. Biocatal. Agr. Biotechnol., 17: 196-202. doi: 10.1016/j.bcab.2018.11.022
Mandinia, E., Demirci, A. & Berenjian, A. (2019e): Evaluation of vitamin K (menaquinone-7) stability and secretion in glucose and glycerol-based media by Bacillus subtilis natto. Acta Alimentaria, 48(4):405-414. doi: 10.1556/066.2019.48.4.1
Pandey A (2003) Solid-state fermentation. Biochem Eng J 13:81-84. doi: 10.1016/S1369-703X(02)00121-3
Pashazanusi L, Lwoya B, Oak S, Khosla T, Albert J N, Tian Y, Bansal G, Kumar N, Pesika NS (2017) Enhanced Adhesion of Mosquitoes to Rough Surfaces. Appl Mater Interfaces 9:24373-24380. doi: 10.1021/acsami.7b06659
Schurgers L J, Teunissen K J, Hamulyák K, Knapen M H, Vik H, Vermeer C (2007) Vitamin K-containing Dietary Supplements: Comparison of Synthetic Vitamin K1 and Natto-derived Menaquinone-7. Blood 109:3279-3283. doi: 10.1182/blood-2006-08-040709
Shahami M, Ransom R, Shantz D F (2017) Synthesis and characterization of tin, tin/aluminum, and tin/boron containing MFI zeolites containing MFI zeolites. Microporous Misoporous Mater 251:165-172. doi: 10.1016/j.micromeso.2017.05.049
Singh R, Puri A, Panda B P (2015) Development of menaquinone-7 enriched nutraceutical: inside into medium engineering and process modeling. J Food Sci Technol 52:5212-5219. doi: 10.1007/s13197-014-1600-7
Stragier P, Bonamy C, Karmazyn-Campelli C (1988) Processing of a Sporulation Sigma Factor in Bacillus subtilis: How Morphological Structure Could Control Gene Expression. Cell 52:697-704. doi: 10.1016/0092-8674(88)90407-2
Stülke J, Hillen W (1999) Carbon catabolite repression in bacteria. Curr Opin Microbiol 2:195-201. doi: 10.1016/S1369-5274(99)80034-4
Walther B, Chollet M, (2017) Menaquinones, bacteria, and foods: Vitamin K2 in the diet. Vitamin K2-Vital for Health and Wellbeing. Intech, pp 63-82. http://dx.doi.org/10.5772/63712
Widhalm J R, Ducluzeau A L, Buller N E, Elowsky C G, Olsen L J, Basset G J (2012) Phylloquinone (vitamin K(1)) biosynthesis in plants: two peroxisomal thioesterases of Lactobacillales origin hydrolyze 1,4-dihydroxy-2-naphthoyl-CoA. Plant J 71:205-215. doi: 10.1111/j.1365-313X.2012.04972.x
Wu W-J, Ahn B-Y (2011) Improved Menaquinone (Vitamin K2) Production in Cheonggukjang by Optimization of the Fermentation Conditions. Food Sci Biotechnol 20:1585-1591. doi: 10.1007/s10068-011-0219-y
Yamaguchi M (2006) Regulatory Mechanism of Food Factors in Bone Metabolism and Prevention of Osteoporosis. Yakugaku Zasshi 126:1117-1137. doi: 10.1248/yakushi.126.1117

1. A method for producing Vitamin K, the method comprising:
i) introducing into a biofilm reactor a first glucose or glycerol containing bacteria culture media, wherein a biofilm comprising bacteria that are capable of producing the Vitamin K forms on a surface in the biofilm reactor, wherein optionally the first glucose or glycerol containing bacteria culture media is replaced one or more times during formation of the biofilm;
ii) introducing a second glycerol or glucose containing bacteria culture media into the biofilm reactor, agitating the bacteria culture medium; and
iii) separating the Vitamin K from the biofilm reactor.

2. The method of claim 1, wherein the first glucose containing bacteria culture media is introduced into the biofilm reactor.

3. The method of claim 2, wherein the first glucose containing bacteria culture media comprises about 150 g/L of the glucose.

4. The method of claim 1, further comprising introducing a second glycerol or glucose containing bacteria culture media into the biofilm reactor, wherein optionally the second glycerol or glucose containing bacteria culture media is introduced at about 72 hours after the introduction of the first glucose containing bacteria culture media.

5. The method of claim 4, comprising introducing the second glycerol containing bacteria culture media into the biofilm reactor.

6. The method of claim 5, wherein the second glycerol containing bacteria culture media comprises about 45 g/L of the glycerol.

7. The method of claim 6, further comprising introducing a third glycerol containing bacteria culture media into the biofilm reactor, wherein optionally the third glycerol containing bacteria culture media is introduced at about 144 hours after the introduction of the first glycerol containing bacteria culture media.

8. The method of claim 7, wherein the third glycerol containing bacteria culture media comprises about 45 g/L of the glycerol.

9. The method of claim 4, comprising introducing the second glucose containing bacteria culture media into the biofilm reactor.

10. The method of claim 9, wherein the second glucose containing bacteria culture media comprises about 150 g/L of the glucose.

11. The method of claim 10, further comprising introducing a third glucose containing bacteria culture media into the biofilm reactor, wherein optionally the third glucose containing media is introduced at about 144 hours after the introduction of the first glucose containing bacteria culture media.

12. The method of claim 1, wherein at least 24 mg/L of the Vitamin K is produced.

13. The method of claim 12, wherein the at least 24 mg/L of the Vitamin K is produced, and wherein optionally said Vitamin K is produced over a time period of not more than about 288 hours from introducing the first glucose or glycerol containing bacteria culture media into the biofilm reactor.

14. The method of claim 13, wherein from 24-30 mg/L of the Vitamin K is produced.

15. The method of claim 14, wherein from 28-29 mg/L of the Vitamin K is produced.

16. The method of claim 15, wherein the agitating is performed continuously over the period of about 288 hours.

17. The method of any one claim 1, wherein the Vitamin K comprises Menaquinone-7 (MK-7).

18. The method of claim 17, wherein the bacteria comprise Bacillus subtilis, and wherein optionally the Bacillus subtilis comprise Bacillus subtilis natto.

19. The method of claim 18, wherein the at least 24 mg/L of the MK-7 is produced over a time period of not more than about 288 hours from introducing the first glucose or glycerol containing bacteria culture media into the biofilm reactor.

20. The method of claim 17, wherein the biofilm reactor comprises one or a plurality of plastic composite supports (PCS) that increase surface area on which the biofilm is formed.

21. A preparation of purified Vitamin K produced according to the method of claim 1.

22. The preparation of purified Vitamin K of claim 21, wherein the Vitamin K comprises Menaquinone-7 (MK-7).

23. A system comprising:
a biofilm reactor and a plurality of plastic composite supports (PCS), the plurality of PCS supports comprising a biofilm comprising bacteria that produce Vitamin K, the system further comprising a bacterial culture media comprising glucose, glycerin, or a combination thereof, wherein the bacterial culture media comprises at least 24-30 mg/L of the Vitamin K.

24. The system of claim 23, wherein the Vitamin K comprises Menaquinone-7 (MK-7).

25. The system of claim 24, wherein the culture media comprises from 24-30 mg/L of the MK-7

26. The system of claim 23, wherein the bacteria in the biofilm comprise Bacillus subtilis, and wherein optionally the Bacillus subtilis comprise Bacillus subtilis natto.
